(19) United States
(12) Patent Application Publication
(10) Pub. No.: US 2009/0042787 A1
(43) Pub. Date: Feb. 12, 2009

(54) Proteolytically cleavable fusion proteins with high molar specific activity

(76) Inventors: Hubert Metzner, Marburg (DE); Thomas Weimer, Gladenbach (DE); Stefan Schulte, Marburg (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/812,016

(22) Filed: Jun. 14, 2007

Related U.S. Application Data
(60) Provisional application No. 60/819,620, filed on Jul. 11, 2006.

(30) Foreign Application Priority Data
Jun. 14, 2006 (EP) 06012262.9

(51) Int. Cl. A61K 38/16; C07K 19/00; C07H 21/04; C12N 15/63; A61P 7/00; A61K 31/711; C12N 5/10; C12P 21/00

(52) U.S. Cl. 514/12; 530/381; 536/23.4; 435/320.1; 435/325; 435/69.7; 514/44

(57) ABSTRACT

The invention relates to therapeutic fusion proteins in which a coagulation factor is fused to a half-life enhancing polypeptide, and in which both are connected by a linker peptide that is proteolytically cleavable. The cleavage of such linkers liberates the coagulation factor from activity-compromising steric hindrance caused by the half-life enhancing polypeptide and thereby allows the generation of fusion proteins may show relatively high molar specific activity when tested in coagulation-related assays. Furthermore, the fact that the linker is cleavable can enhance the rates of inactivation and/or elimination after proteolytic cleavage of the peptide linker compared to the rates measured for corresponding therapeutic fusion proteins linked by the non-cleavable linker having the amino acid sequence GGGGGGV (SEQ ID NO: 94).

This application claims priority to U.S. Provisional Application No. 60/819,620, filed Jul. 11, 2006, and also claims priority to European Patent Application No. 06012262.9, filed Jun. 14, 2006.

The present invention relates to the field of modified therapeutic fusion proteins with increased half-life compared to their non-modified parent therapeutic polypeptides. The invention, for example, relates to coagulation factors fused to half-life enhancing polypeptides (HLEPs), which are connected by linker peptides that are proteolytically cleavable. The cleavage of such linkers liberates the therapeutic polypeptide from activity-compromising steric hindrance caused by the HLEP and thereby allows the generation of fusion proteins, which retain a high molar specific activity of the coagulation factor. When the therapeutic fusion proteins are zymogens, one may use linkers that liberate the therapeutic polypeptide essentially simultaneously with its activation in vivo upon exposure to the corresponding protease(s). Embodiments of the present invention may show a faster inactivation rate of a given coagulation factor once the coagulation factor is activated and the peptide linker is proteolytically cleaved in a coagulation-related mode and/or a faster elimination rate of a given coagulation factor once the coagulation factor is activated and the peptide linker is proteolytically cleaved in a coagulation-related mode, compared to the corresponding fusion protein without cleavable linker.

One aspect of the invention is demonstrated for example by human vitamin K-dependent polypeptides Factor IX, Factor VII, and Factor VIIa. The same concept also may be applied to other coagulation factors. Any half-life enhancing polypeptide (HLEP) may be connected to the therapeutic polypeptide by a cleavable linker peptide. For instance albumin or immunoglobulins or fragments derived therefrom without an antigen binding domain, such as the Fc fragment, may serve as HLEPs. The invention also relates to cDNA sequences coding for the therapeutic polypeptides and derivatives thereof genetically fused to a cDNA coding for HLEPs, such as human serum albumin linked by oligonucleotides that code for cleavable, intervening peptide linkers. Such encoded derivatives may exhibit improved half-life and molar specific activities that are increased in comparison to their non-cleavable counterparts. The invention also relates to recombinant expression vectors containing such cDNA sequences, host cells transformed with such recombinant expression vectors, recombinant polypeptides and derivatives which may have biological activities comparable to the unmodified wild type therapeutic polypeptide, but having improved half-lifes. The invention also relates to processes for the manufacture of such recombinant proteins and their derivatives. The invention also relates to a transfer vector for use in human gene therapy, which comprises such modified DNA sequences, which can be useful to increase half-life in vivo.

Several recombinant, therapeutic polypeptides are commercially available for therapeutic and prophylactic use in humans. The patients in general may benefit from the specific mode of action of the recombinant active ingredients but a disadvantage may be their limited availability due to expensive and complex manufacturing processes. A reduction of the necessary dose or the frequency of administration of such products could improve this situation. A reduced frequency of administration could improve the convenience for the patient and, therefore, also the acceptance of the therapy. Several ideas have been described to attempt to achieve the goal of an increased in vivo half-life after administration. See, e.g. Balance et al. (WO 01/79271), Sheffield et al. (Sheffield W. P. et al. (2004), Br. J. Haematol. 126: 565-573), WO 2002/04598, WO 2003/059935, WO 2004/081053, WO 2004/101740, WO 2005/001025, WO 91/09125, and WO 03/068934.

Fusions of coagulation factors to half-life enhancing polypeptides have been suggested to lengthen the half-life of coagulation factors administered to patients. However, once a coagulation factor is activated during coagulation either by proteolytic cleavage of the zymogen (like FIX) or by contact of an already proteolytically “pre”-activated factor to a second polypeptide (like FVIIa binding to Tissue Factor), it may no longer be desirable to maintain the long half-life of the now activated coagulation factor, as this might lead to thrombotic complications. This is the case for a wild type coagulation factor such as FVIIa (Aledort L. M., J Thromb Haemost 2(10): 1700-1708 (2004)) and may be even more relevant if the activated factor has an increased half-life. It is therefore one objective of some emobodiments of the present invention to provide long-lived coagulation factors, which after activation or after availability of a cofactor have a half-life comparable to that of an unmodified coagulation factor.

Fusions of the coagulation factors to half-life enhancing polypeptides as described in the prior art and as also shown in examples 6 and 7 may suffer in general from a reduced molar specific activity of the fused coagulation factor. Another aspect of the present invention is to provide coagulation factors with enhanced half-life that show increased molar specific activity compared to the corresponding therapeutic fusion protein without a cleavable linker.

Some embodiments of this invention include therapeutic fusion proteins comprising:
a) a coagulation factor,
b) a half-life enhancing polypeptide (HLEP) chosen from albumin and immunoglobulin, and
c) a peptide linker, which linker joins the coagulation factor and the half-life enhancing polypeptide;
   wherein the peptide linker is cleavable by proteases involved in coagulation or proteases activated by coagulation enzymes, and wherein the therapeutic fusion protein has, in comparison to the respective therapeutic fusion protein linked by a non-cleavable linker having the amino acid sequence GGGGGGV, at least one of the following properties:
i) an increased molar specific activity in at least one coagulation-related assay,
ii) an increased inactivation rate of the activated coagulation factor after the peptide linker is proteolytically cleaved in a coagulation-related mode, and
iii) an increased elimination rate of the activated coagulation factor after the peptide linker is proteolytically cleaved in a coagulation-related mode.

A “coagulation factor,” as used herein, includes variants or derivatives thereof, such as genetically engineered or chemically modified variants or active fragments thereof. See below for additional description of examples of “coagulation factors.”

“Albumin,” as used herein, includes polypeptides of the albumin family of proteins such as human serum albumin and bovine serum albumin, including variants and derivatives thereof, such as genetically engineered or chemically modified albumin variants and fragments of albumin proteins. See below for additional description of examples of “albumin.”

“Immunoglobulin,” as used herein, includes variants and derivatives of immunoglobulin proteins, such as genetically engineered or chemically modified immunoglobulin variants and fragments of immunoglobulin, for example, an Fc fragment or other fragment not containing an antigen binding domain. See below for additional description of examples of “immunoglobulin.”

As a consequence of the cleavable linker, after cleavage of the peptide linker in a coagulation-related mode the coagulation factor more closely resembles the behaviour of the native, non-fused factor and does not show an increased half-life of the active factor with potentially prothrombotic effect.

“Proteolytic cleavage in a coagulation-related mode,” as used herein, means any proteolytic cleavage that occurs as a consequence of the activation of at least one coagulation factor or coagulation cofactor.

The phrase “activated coagulation factor after the peptide linker is proteolytically cleaved in a coagulation-related mode,” as used herein means that the coagulation factor is either activated almost in parallel to the proteolytic cleavage of the linker peptide, or that the coagulation factor was already activated before the proteolytic cleavage of the linker peptide. Activation may occur, for example by proteolytic cleavage of the coagulation factor or by binding to a cofactor.

A further aspect of the present invention is to provide therapeutic fusion proteins comprising:
a) a coagulation factor,
b) a half-life enhancing polypeptide (HLEP) chosen from albumin and immunoglobulin, and
c) a peptide linker which joins the coagulation factor and the half-life enhancing polypeptide;
   wherein the peptide linker is cleavable by proteases involved in coagulation or activated by coagulation enzymes, and wherein the therapeutic fusion protein has, in comparison to the respective therapeutic fusion protein linked by a non-cleavable linker having the amino acid sequence GGGGGGV, at least one of the following properties:
i) an increased molar specific activity in at least one coagulation-related assay,
ii) an increased inactivation rate of the activated coagulation factor after the peptide linker is proteolytically cleaved in a coagulation-related mode,
iii) an increased elimination rate of the activated coagulation factor after the peptide linker is proteolytically cleaved in a coagulation-related mode, and
iv) an enhanced in vivo recovery as compared to the in vivo recovery of the unmodified coagulation factor.

Some embodiments include therapeutic fusion proteins which have an enhanced in vivo recovery compared to the unmodified coagulation factor by at least 10%, for example, by at least 25% or by 40% or more.

Exemplary coagulation factors are vitamin-K dependent coagulation factors, FVIIa, and FIX, and fragments and variants thereof, such as genetically engineered or chemically modified variants or fragments, such as are described in more detail below.

HLEPs may be albumin and fragments or variants thereof and immunoglobulins including fragments and variants thereof, as described above, and in more detail, below.

The peptide linker in some embodiments may comprise a sequence of the therapeutic polypeptide to be administered or a variant thereof, which should result in a decreased risk of neoantigenic properties (formation of a novel potentially immunogenic epitope due to the occurrence of a peptide within the therapeutic antigen which does not exist in human proteins) of the expressed fusion protein. Also in case the therapeutic protein is a zymogen (e.g. needs to be proteolytically activated) the kinetics of the peptide linker cleavage may more closely reflect the coagulation-related activation kinetics of the zymogen. Thus, in some embodiments, a zymogen and a corresponding peptide linker are activated and respectively cleaved, with comparable kinetics. For this reason, embodiments of the present invention also relate to fusion proteins of a zymogen and a HLEP, where the kinetics of the linker cleavage by relevant proteases are not delayed by more than a factor of 3, such as not by more than a factor of 2, compared to the kinetics of the zymogen activation.

In another embodiment, the peptide linker comprises cleavage sites for more than one protease. This can be achieved, for example, by a peptide linker that can be cleaved at the same position by different proteases or by a peptide linker that provides two or more different cleavage sites. This may be advantageous circumstances where the therapeutic fusion protein must be activated by proteolytic cleavage to achieve enzymatic activity and where different proteases may contribute to this activation step. This is the case, for example, upon activation of FIX, which can either be achieved by FXIa or by FVIIa/Tissue Factor (TF).

Some embodiments of the invention are therapeutic fusion proteins wherein the peptide linker is cleavable by the protease that normally activates the coagulation factor in vivo, thereby ensuring that the cleavage of the linker is linked to the activation of the coagulation factor at a site at which coagulation occurs.

Other exemplary therapeutic fusion proteins according to the invention are those wherein the linker is cleavable by the coagulation factor which is part of the therapeutic fusion protein once it is activated, thus also ensuring that cleavage of the fusion protein is connected with a coagulatory event.

Other exemplary therapeutic fusion proteins according to the invention are those wherein the linker is cleavable by a protease, which itself is activated directly or indirectly by the activity of the coagulation factor which is part of the therapeutic fusion protein, thus also ensuring that cleavage of the fusion protein is connected with a coagulatory event.

One class of therapeutic fusion proteins included in this invention comprises those wherein the linker is cleavable by FXIa and/or by FVIIa/TF and the coagulation factor is FIX.

For example, embodiments of the invention include fusion proteins comprising the vitamin K-dependent polypeptide Factor IX, cleavable linkers, and albumin as the HLEP, as well as their corresponding cDNA sequences. The invention also relates to cDNA sequences coding for any other coagulation factors which can be proteolytically activated or that are involved in the activation of other zymogens or polypeptides. In some embodiments, these cDNAs are genetically fused to cDNA sequences coding for human serum albumin or other HLEPs, and are linked by oligonucleotides that code for intervening, cleavable peptide linkers. The expressed therapeutic fusion proteins may exhibit molar specific activities which are increased in comparison to their non-cleavable counterparts. The invention also relates to recombinant expression vectors containing such fused cDNA sequences, host cells transformed with such recombinant expression vectors, recombinant therapeutic fusion proteins and derivatives that may have biological activities almost comparable to the unmodified wild type therapeutic polypeptides but having improved in vivo half-life. The invention also relates to processes for the manufacture of the recombinant polypeptides of the invention and their derivatives. The invention also relates to a transfer vector for use in human gene therapy, which comprises such modified DNA sequences useful to increase product levels in vivo.

Some therapeutic fusion proteins according to the invention have a molar specific activity, in particular a molar specific activity in at least one coagulation-related assay, that is at least 25% increased compared to that of the corresponding therapeutic fusion protein without a cleavable linker. Other therapeutic fusion proteins have a molar specific activity that is increased by at least 50%, or by at least 100%, in at least one of the different coagulation-related assays available, compared to that of the corresponding therapeutic fusion protein without a cleavable linker, such as one with the non-cleavable sequence GGGGGGV.

Additional embodiments of the present invention are therapeutic fusion proteins wherein the inactivation rate of the activated coagulation factor after cleavage of the peptide linker which links the coagulation factor to the half-life enhancing polypeptide is increased by at least 10% as compared to the inactivation rate of the activated coagulation factor in a corresponding therapeutic fusion protein without a cleavable linker. Other embodiments are therapeutic fusion proteins in which the inactivation rate is increased by at least 25%, or by at least 50% as compared to the inactivation rate of the activated coagulation factor in a corresponding therapeutic fusion protein without a cleavable linker, such as one with the non-cleavable sequence GGGGGGV.

Additional embodiments of the present invention are therapeutic fusion proteins wherein the elimination rate of the coagulation factor after cleavage of the peptide linker that links the coagulation factor to the half-life enhancing polypeptide is increased by at least 10% as compared to the elimination rate of the coagulation factor in a corresponding therapeutic fusion protein without a cleavable linker. Other embodiments are therapeutic fusion proteins in which the elimination rate is increased by at least 25%, or by at least 50% as compared to the elimination rate of the coagulation factor in a corresponding therapeutic fusion protein without a cleavable linker, such as one with the non-cleavable sequence GGGGGGV.

### Vitamin K-Dependent Polypeptides

Vitamin K-dependent polypeptides as one group of the therapeutic polypeptides are polypeptides that are γ-carboxylated enzymatically in the liver using vitamin K as a cofactor. Such vitamin K-dependent polypeptides e.g. include Factors II, VII, IX, X, Protein C, Protein S, GAS6, and Protein Z.

### Human FIX

Human FIX, one member of the group of vitamin K-dependent polypeptides, is a single-chain glycoprotein with a molecular weight of 57 kDa, which is secreted by liver cells into the blood stream as an inactive zymogen of 415 amino acids. It contains 12 γ-carboxy-glutamic acid residues localized in the N-terminal Gla-domain of the polypeptide. The Gla residues require vitamin K for their biosynthesis. Following the Gla domain there are two epidermal growth factor domains, an activation peptide, and a trypsin-type serine protease domain. Further posttranslational modifications of FIX encompass hydroxylation (Asp 64), N-(Asn157 and Asn167) as well as O-type glycosylation (Ser53, Ser61, Thr159, Thr169, and Thr172), sulfation (Tyr155), and phosphorylation (Ser158). FIX is converted to its active form, Factor IXa, by proteolysis of the activation peptide at Arg145-Ala146 and Arg180-Val181 leading to the formation of two polypeptide chains, an N-terminal light chain (18 kDa) and a C-terminal heavy chain (28 kDa), which are held together by one disulfide bridge. Activation cleavage of Factor IX can be achieved in vitro e.g. by Factor XIa or Factor VIIa/TF. Factor IX is present in human plasma in a concentration of 5-10 μg/ml. Terminal plasma half-life of Factor IX in humans was found to be about 15 to 18 hours (White G C et al. 1997. Recombinant factor IX. Thromb Haemost. 78: 261-265; Ewenstein B M et al. 2002. Pharmacokinetic analysis of plasma-derived and recombinant F IX concentrates in previously treated patients with moderate or severe hemophilia B. Transfusion 42:190-197).

Hemophilia B is caused by non-functional or missing Factor IX and is treated with Factor IX concentrates from plasma or a recombinant form of Factor IX. As haemophilia B patients often receive at least biweekly prophylactic administrations of Factor IX to avoid spontaneous bleedings, it is desirable to increase the intervals of time between administrations by increasing the half-life of the Factor IX product applied. An improvement in plasma half-life may bring significant benefit to the patient. Up to now no pharmaceutical preparation of a Factor IX with improved plasma half-life is commercially available nor have any data been published showing F IX variants with prolonged in vivo half-life and almost unchanged molar specific activity in coagulation-related assays. Therefore, a great medical need still exists to develop forms of Factor IX which have a longer functional half-life in vivo.

### Factor VII and Factor VIIa

FVII is a single-chain glycoprotein with a molecular weight of 50 kDa, which is secreted by liver cells into the blood stream as an inactive zymogen of 406 amino acids. FVII is converted to its active form Factor VIIa, by proteolysis of the single peptide bond at Arg152-IIe 153 leading to the formation of two polypeptide chains, a N-terminal light chain (24 kDa) and a C-terminal heavy chain (28 kDa), which are held together by one disulfide bridge. In contrast to other vitamin K-dependent coagulation factors, no activation peptide is cleaved off during activation. Activation cleavage of Factor VII can be achieved in vitro, for example, by Factor Xa, Factor IXa, Factor VIIa, Factor XIIa, Factor Seven Activating Protease (FSAP), and thrombin. Mollerup et al. (Biotechnol. Bioeng. (1995) 48: 501-505) reported that some cleavage also occurs in the heavy chain at Arg290 and/or Arg315.

Factor VII is present in plasma in a concentration of 500 ng/ml. About 1% or 5 ng/ml of Factor VII is present as activated Factor VIIa. The terminal plasma half-life of Factor VII was found to be about 4 hours and that of Factor VIIa about 2 hours.

By administering supraphysiological concentrations of Factor VIIa hemostasis can be achieved bypassing the need for Factor VIIIa and Factor IXa. The cloning of the cDNA for Factor VII (U.S. Pat. No. 4,784,950) made it possible to develop activated Factor VII as a pharmaceutical. Factor VIIa was successfully administered for the first time in 1988. Ever since the number of indications of Factor VIIa has grown steadily showing a potential to become an universal hemostatic agent to stop bleeding (Erhardtsen, 2002). However, the short terminal half-life of Factor VIIa of approximately 2 hours and reduced in vivo recovery may be limiting its application. Therefore, a great medical need still exists to develop forms of Factor VIIa which have an improved half-life but otherwise almost uncompromised molar specific activity, inactivation kinetics, and/or elimination kinetics after start of coagulation.

### Therapeutic Fusion Proteins

“Therapeutic fusion proteins” or “fusion proteins” as used herein are coagulation factors fused to a half-life enhancing polypeptide such that, upon administration to a human or other animal, may produce a prophylactic or therapeutic effect. These therapeutic fusion proteins may be administered to a human or other animal via, for example, intravenous, intramuscular, subcutaneous, oral, topical, parenteral or other routes. In addition, gene therapy protocols may be used which involve administration of a polynucleotide encoding the fusion protein or a composition comprising that polynucleotide, such as a plasmid or vector or host cell. Specific classes of therapeutic fusion proteins described, i.e. by the examples below, are coagulation factors, such as vitamin K-dependent polypeptides, linked to half-life enhancing polypeptides, such as albumin and immunoglobulin. The expression “therapeutic fusion protein” is used interchangeably with “fusion protein”.

### Half-Life Enhancing Polypeptide (HLEP)

Albumin and immunoglobulin have been described above as examples of half-life enhancing polypeptides (HLEPs). The terms “human serum albumin” (HSA) and “human albumin” (HA) are used interchangeably in this application. The terms “albumin” and “serum albumin” are broader, and encompass human serum albumin (and fragments and variants thereof) as well as albumin from other species (and fragments and variants thereof).

“Albumin,” as used herein, includes polypeptides of the albumin family of proteins. As used herein, “albumin” refers collectively to albumin polypeptide or amino acid sequence, or an albumin fragment or variant having one or more functional activities (e.g., biological activities) of albumin. Examples include human serum albumin and bovine serum albumin, including variants and derivatives thereof, such as genetically engineered or chemically modified albumin variants and fragments of albumin proteins. For example, “albumin” refers to human albumin or fragments thereof, such as the mature form of human albumin as shown in SEQ ID No:1 herein or albumin from other vertebrates or fragments thereof, or analogs or variants of these molecules or fragments thereof.

The albumin portion of the albumin fusion proteins may comprise the full length of, for instance, the HA sequence as described above, or may include one or more fragments thereof that are capable of stabilizing or prolonging the therapeutic activity. Such fragments may be of 10 or more amino acids in length or may include about 15, 20, 25, 30, 50, or more contiguous amino acids from the HA sequence or may include part or all of specific domains of HA.

The albumin portion of the albumin fusion proteins of the invention may comprise a variant or derivative or analog of normal HA, either natural or artificial. The therapeutic polypeptide portion of the fusion proteins of the invention may also comprise variants of the corresponding therapeutic polypeptides as described herein. The terms “variants” “derivatives” and “analogs” each include, for example, insertions, deletions, and substitutions, either conservative or non-conservative, either natural or artificial (i.e. engineered), where such changes do not substantially alter the active site, or a fragment, such as an active domain that confers the therapeutic activities of the therapeutic polypeptides, or chemical modifications that also allow for therapeutic activity.

In particular, the albumin fusion proteins of the invention may include naturally occurring polymorphic variants of human albumin and fragments of human albumin. The albumin may be derived from any vertebrate, especially any mammal, for example human, cow, sheep, or pig. Non-mammalian albumins include, but are not limited to, hen and salmon. The albumin portion of the albumin-linked polypeptide may be from a different animal than the therapeutic polypeptide portion.

Generally speaking, an albumin fragment or variant encompassed within the term “albumin” will be at least 10, for example at least 40, or more than 70 amino acids long. That albumin variant may comprise at least one whole domain of albumin or fragments of said domains, for example domains 1 (amino acids 1-194 of SEQ ID NO:1), 2 (amino acids 195-387 of SEQ ID NO:1), 3 (amino acids 388-585 of SEQ ID NO:1), 1+2 (1-387 of SEQ ID NO:1), 2+3 (195-585 of SEQ ID NO:1) or 1+3 (amino acids 1-194 of SEQ ID NO:1+amino acids 388-585 of SEQ ID NO:1). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315, and Glu492 to Ala511.

The albumin portion of an albumin fusion protein of the invention may comprise at least one subdomain or domain of HA or conservative modifications thereof.

All fragments and variants of albumin are encompassed by the invention as fusion partners of a coagulation factor as long as they lead to a half-life extension of the therapeutic fusion protein in plasma of at least 25% as compared to the non-fused coagulation factor.

The albumin family of proteins, included within the term “albumin” used herein, comprise evolutionarily related serum transport proteins, for example, albumin, alpha-fetoprotein (AFP; Beattie & Dugaiczyk 1982. Gene 20:415-422), afamin (AFM; Lichenstein et al. 1994. J. Biol. Chem. 269:18149-18154) and vitamin D binding protein (DBP; Cooke & David 1985. J. Clin. Invest. 76:2420-2424). Alpha-fetoprotein has been claimed to enhance the half-life of an attached therapeutic polypeptide in vivo (WO 2005/024044). Their genes represent a multigene cluster with structural and functional similarities mapping to the same chromosomal region in humans, mice and rat. The structural similarity of those albumin family members suggests their usability as HLEPs. Some embodiments of the invention, therefore, may use such albumin family members, or fragments and variants thereof as defined above, as HLEPs.

Albumin family members encompassed within the term “albumin” herein also comprise the full length of the respective proteins AFP, AFM and DBP, or may include one or more fragments thereof that are capable of stabilizing or prolonging the therapeutic activity. Such fragments may be of 10 or more amino acids in length or may include about 15, 20, 25, 30, 50, or more contiguous amino acids of the respective protein sequence or may include part or all of specific domains of the respective protein, as long as the HLEP fragments provide a half-life extension of at least 25% as compared to the non-fused coagulation factor. Albumin family members of the therapeutic fusion proteins of the invention may also include naturally occurring polymorphic variants of AFP, AFM and DBP.

The term “immunoglobulin” as used herein, encompasses, for instance, IgG and IgG-fragments, which may also be used as HLEPs, as long as the corresponding HLEP fragments provide a half-life extension of at least 25% as compared to the non-fused coagulation factor. The therapeutic polypeptide portion may be connected to the IgG or the IgG fragments via a cleavable linker that allows high molar specific activities of the fusion protein. For example, fusion proteins comprising FVII or VIIa and IgG or fragments thereof may be prepared. A linker sequence of the present invention liberating FVII (FVIIa) molecules upon cleavage by a protease of the coagulation cascade such as, e.g., FXIa, FXa, or FIXa could be able to elevate the clotting activity of the constructs to an activity level comparable to the monomer/dimer or even higher. A FIX-Fc fusion protein with cleavable linker is exemplarily shown in SEQ ID NO:93. Cleavable linkers such as those shown in table 3a and 3b may also be applied in such embodiments.

The invention also relates to fusion proteins comprising linking a coagulation factor, including, for example, a fragment or variant thereof, to the N- or C-terminus of a HLEP, including a fragment or variant thereof, such that an intervening cleavable peptide linker is introduced between the therapeutic polypeptide and the HLEP such that the fusion protein formed has an increased in vivo half-life compared to the coagulation factor which has not been linked to a HLEP and that the fusion protein has an at least 25% higher molar specific activity compared to the corresponding fusion protein with non-cleavable linker in at least one of the different coagulation-related assays available.

“Coagulation factor” as used in this application includes, but is not limited to, polypeptides consisting of Factor IX, Factor VII, Factor VIII, von Willebrand Factor, Factor V, Factor X, Factor XI, Factor XII, Factor XIII, Factor I, Factor II (Prothrombin), Protein C, Protein S, GAS6, or Protein Z as well as their activated forms. A “coagulation factor,” as used herein, also includes variants or derivatives thereof, such as genetically engineered or chemically modified variants or active fragments thereof. For instance, useful therapeutic polypeptides may be wild-type polypeptides or may contain mutations. Degree and location of glycosylation or other post-translation modifications may vary depending on the chosen host cells and the nature of the host cellular environment. When referring to specific amino acid sequences, posttranslational modifications of such sequences are encompassed in this application.

“Coagulation factor” within the above definition includes polypeptides that have the natural amino acid sequence including any natural polymorphisms. It also includes polypeptides with a slightly modified amino acid sequence, for instance, a modified N-terminal or C-terminal end including terminal amino acid deletions or additions, as long as those polypeptides substantially retain the activity of the respective therapeutic polypeptide. Variants included may differ in one or more amino acid residues from the wild type sequence. Examples of such differences may include truncation of the N- and/or C-terminus by one or more amino acid residues (e.g. preferably 1 to 30 amino acid residues), or addition of one or more extra residues at the N- and/or C-terminus, as well as conservative amino acid substitutions, i.e. substitutions performed within groups of amino acids with similar characteristics, e.g. (1) small amino acids, (2) acidic amino acids, (3) polar amino acids, (4) basic amino acids, (5) hydrophobic amino acids, and (6) aromatic amino acids. Examples of such conservative substitutions are shown in the following table.

TABLE 1

| | | | | |
|---|---|---|---|---|
| (1) | Alanine | Glycine | | |
| (2) | Aspartic acid | Glutamic acid | | |
| (3a) | Asparagine | Glutamine | | |
| (3b) | Serine | Threonine | | |
| (4) | Arginine | Histidine | Lysine | |
| (5) | Isoleucine | Leucine | Methionine | Valine |
| (6) | Phenylalanine | Tyrosine | Tryptophane | |

The in vivo half-life of the fusion proteins of the invention, in general determined as terminal half-life or β-half-life, may be at least about 25%, at least about 50%, or more than 100% higher than the in vivo half-life of the non-fused polypeptide.

The fusion proteins of the present invention also have at least a 25%, and may have at least a 50% or at least 100% increased molar specific activity compared to the corresponding fusion proteins without cleavable linkers.

The “molar specific activity” (or molar specific coagulation-related activity as considered here in particular) in this regard is defined for purposes herein as the activity expressed per mole (or e.g. nmole) of the therapeutic polypeptide or therapeutic fusion protein of interest. Calculation of the molar specific activity allows a direct comparison of the activity of the different constructs that is not affected by the different molecular weights or optical densities of the polypeptides studied. The molar specific activity may be calculated as exemplified in table 2 below for FIX and a FIX-HSA fusion protein.

TABLE 2
Calculation of molar specific activity as shown for a purified FIX-HSA
fusion protein

| | | | | Molar optical | Calculation of molar |
|---|---|---|---|---|---|
| | | | Activity/Vol/OD₂₈₀ | density (OD₍₂₈₀₎ | specific activity |
| Product | OD_{(280 nm, 1%)} | MW | (IU/L/OD₂₈₀) | at 1 mol/L) | (IU/mol) |
| FIX | 13.3 ¹⁾ | 57 000 | determined for | 75810 (=MW × | =(Activity/Vol/OD₂₈₀) × |
| | | | product | OD_{(280 nm, 1%)}/10) | (OD₂₈₀ at 1 mol/L) |
| HSA | 5.7 ²⁾ | 66 300 | | 37791 (=MW × | |
| | | | | OD_{(280 nm, 1%)}/10) | |
| FIX-HSA | | | determined for | 113601 (=sum | =(Activity/Vol/OD₂₈₀) × |
| | | | product | of molar optical | (OD₂₈₀ at 1 mol/L) |
| | | | | density of FIX | |
| | | | | and HSA) | |

[symbol]

Di Scipio et al., Biochem. 16: 698-706 (1977) [symbol] [symbol]
et al, J. Exp. Med. 197(3): 315-322 (2003)

In order to determine a molar specific coagulation-related activity, any assay may be used that determines enzymatic or cofactor activities that are relevant to the coagulation process.

Therefore “coagulation-related assays” in the sense of the invention comprise any assay which determines enzymatic or cofactor activities that are of relevance in the coagulation process or that are able to determine that either the intrinsic or the extrinsic coagulation cascade has been activated. The “coagulation-related” assay thus may be direct coagulation assays like aPTT, PT, or the thrombin generation assays. However, other assays such as chromogenic assays applied for specific coagulation factors are also included. Examples of such assays or corresponding reagents are Pathromtin® SL (aPTT assay, Dade Behring) or Thromborel® S (Prothrombin time assay, Dade Behring) with corresponding coagulation factor deficient plasma (Dade Behring), Thrombin generation assay kits (Technoclone™, Thrombinoscope™) using e.g. coagulation factor deficient plasma, chromogenic assays like Biophen™ Factor IX (Hyphen BioMed), Staclot® FVIIa-rTF (Roche Diagnostics GmbH), Coatest® Factor VIII:C/4 (Chromogenix), or others.

For purposes of this invention, an increase in any one of the above assays or an equivalent coagulation-related assay is considered to show an increase in molar specific activity. For example, a 25% increase refers to a 25% increase in any of the above or an equivalent assay.

To determine whether therapeutic fusion proteins fall within the scope of the present invention, the standard against which the molar specific activity of these proteins is compared is a construct in which the respective coagulation factor and the respective HLEP are linked by a non-cleavable linker having the amino acid sequence GGGGGGV.

In the case of FIX, aPTT assays are often used for determination of coagulation activity. Such a coagulation assay (aPTT assay) is described in example 5 in more detail. However, other coagulation-related assays or assay principles may be applied to determine molar specific activity for FIX.

Recombinant therapeutic polypeptide drugs are usually expensive and not all countries can afford costly therapies based on such drugs. Increasing the in vivo recovery of such drugs could make the use of these products cheaper and subsequently more patients would benefit from them. In the case of the fusion proteins of the present invention an increased in vivo recovery would also be a desirable advantage. “In vivo recovery” as used herein in the sense of the invention means the amount of product found in blood or plasma shortly after administration of the product. For detection of the in vivo recovery in general the plasma content is determined a few minutes (e.g. 5 or 15 min) after administration of the product.

Although it is desirable to have a high in vivo recovery and a long half-life for a non-activated coagulation factor, it is advantageous to limit the half-life of a coagulation factor after its activation or the activation of its co-factor in order to avoid a prothrombotic risk. Therefore, after the coagulation process has been initiated, the half-life of the active coagulation factor should again be reduced. This can either be achieved by enhancing inactivation in a coagulation-related mode or by elimination of the coagulation factor.

“Inactivation” according to the present invention means the decrease of activity of the therapeutic polypeptide which can be caused, for example, by a complex formation of a coagulation factor and an inhibitor of the corresponding coagulation factor or by further proteolytic cleavage as known, e.g., in the case of FVIII and FV.

The “inactivation rate” of an activated therapeutic fusion protein is defined as the rate the activity is declining, e.g., by reaction with inhibitors or by proteolytic inactivation. The inactivation rate may be measured by following the molar specific activity of the activated coagulation factor over time in the presence of physiologic amounts of inhibitors of this coagulation factor. Alternatively, the inactivation rate may be determined after administration of the activated product to an animal followed by testing of plasma samples at an appropriate time frame using activity and antigen assays.

To determine whether therapeutic fusion proteins fall within the scope of the present invention, the standard against which the molar specific activity of these proteins is compared is a construct in which the respective coagulation factor and the respective HLEP are linked by a non-cleavable linker having the amino acid sequence GGGGGGV.

The “elimination rate” of an activated therapeutic fusion protein is defined as the rate the polypeptide is eliminated from the circulation of humans or other animals. The elimination rate may be determined by measuring the pharmacokinetics of the activated, therapeutic fusion protein after intravenous administration. Using an antigen assay, the elimination by direct removal from the circulation can be determined. Using an activity assay in addition, a specific removal and inactivation rate may be determined.

To determine whether therapeutic fusion proteins fall within the scope of the present invention, the standard against which the molar specific activity of these proteins is compared is a construct in which the respective coagulation factor and the respective HLEP are linked by a non-cleavable linker having the amino acid sequence GGGGGGV.

According to some embodiments of this invention, the therapeutic polypeptide moiety is coupled to the HLEP moiety by a cleavable peptide linker. The linker should be non-immunogenic and should be flexible enough to allow cleavage by proteases. The cleavage of the linker should proceed comparably fast as the activation of the therapeutic polypeptide within the fusion protein, if the fusion protein is a zymogen.

The cleavable linker may comprise a sequence derived from
a) the therapeutic polypeptide to be administered itself if it contains proteolytic cleavage sites that are proteolytically cleaved during activation of the therapeutic polypeptide,
b) a substrate polypeptide of this therapeutic polypeptide, or
c) a substrate polypeptide cleaved by a protease which is activated or formed by the direct or indirect involvement of the therapeutic polypeptide.

The peptide linker in some embodiments comprises a sequence of the therapeutic polypeptide to be applied, which should result in a decreased risk of neoantigenic properties of the expressed fusion protein. Also in case the therapeutic protein is a zymogen (e.g. needs to be proteolytically activated) the kinetics of the peptide linker cleavage will more closely reflect the coagulation-related activation kinetics of the zymogen in some embodiments.

In some embodiments, the therapeutic polypeptide is FIX zymogen and the HLEP is albumin. In some of those embodiments the linker sequence is either derived from the sequences of the activation regions of FIX, from the cleavage region of any substrate of FIX like FX or FVII or from the cleavage region of any substrate polypeptide that is cleaved by a protease in whose activation FIXa is involved.

In yet other embodiments, the linker peptide is derived from FIX itself. In other embodiments the linker peptide is derived from FX or FVII. In other embodiments the linker sequence comprises two cleavage sequences that can be cleaved by FXIa or FVIIa/TF, two physiologically relevant activators of FIX.

Exemplary combinations of therapeutic polypeptide, peptide linker, and HLEP include the constructs listed in tables 3a and 3b, but are not limited to them:

TABLE 3a
Examples of possible constructs

| | | | | |
|---|---|---|---|---|
| | | | Linker | |
| | | | derived | |
| | | | from (with | SEQ |
| Coagulation | | | modifications | ID |
| factor | Linker | HLEP | if applicable) | NO: |

Linker not cleavable or not sufficiently rapidly cleavable

| | | | |
|---|---|---|---|
| FIX | — | HSA | |
| FIX | RI | HSA | |
| FIX | GGGGGGV | HSA | 94 |
| | (Sheffield at al.) | | |
| FIX | (GGS)nGS | HSA | |
| FIX | SS(GGS)₇GS | HSA | 30 |
| FIX | SSNGS(GGS)₃NGS(GGS)₃GGNGS | HSA | 31 |

Linker with one cleavage site

| | | | | |
|---|---|---|---|---|
| FIX (1-412) | SVSQTSKLTRAETVFPDVD | HSA | FIX | 36 |
| FIX (1-412) | SVSQTSKLTRAETVFPDVD GS | HSA | FIX | 37 |
| FIX | SVSQTSKLTRAETVFPDVD | HSA | FIX | 38 |
| FIX | SVSQTSKLTRAETVFPDVD GS GGS | HSA | FIX | 95 |
| FIX | SVSQTSKLTRAETVFPDVD GS | HSA | FIX | 39 |
| FIX | SVSQTSKLTRAETVFPDVD NGS | HSA | FIX | 40 |
| FIX | SVSQTSKLTRAETVFPDV | HSA | FIX | 96 |
| FIX | QTSKLTRAETVFPDV | HSA | FIX | 97 |
| FIX | SKLTRAETVFPDV | HSA | FIX | 98 |
| FIX | SVSQTSKLTRAETVFP | HSA | FIX | 99 |
| FIX | SVSQTSKLTRAETVF | HSA | FIX | 100 |
| FIX | QTSKLTRAETVF | HSA | FIX | 101 |
| FIX | SKLTRAETVF | HSA | FIX | 102 |
| FIX | SVSQTSKLTRAET | HSA | FIX | 103 |
| FIX | QTSKLTRAET | HSA | FIX | 104 |
| FIX | SKLTRAET | HSA | FIX | 105 |
| FIX | SVSQTSKLTRGETVFPDVD | HSA | FIX | 41 |
| FIX | SVSQTSKLTRTETVFPDVD | HSA | FIX | 42 |
| FIX | SVSQTSKLTRSETVFPDVD | HSA | FIX | 43 |
| FIX | SVSQTSKLTRLETVFPDVD | HSA | FIX | 44 |
| FIX | SVSQTSKLTRTEAVFPDVD | HSA | FIX | 45 |
| FIX | SVSQTSKLTRGEAVFPDVD | HSA | FIX | 46 |
| FIX | QTSKLTRAETVFPDVD GS | HSA | FIX | 106 |
| FIX | SKLTRAETVFPDVD GS | HSA | FIX | 107 |
| FIX | SKLTRAETVFPDVD | HSA | FIX | 47 |
| FIX | QSFNDFTRVVGGED | HSA | FIX | 48 |
| FIX | QSFNDFTRVVGGED GS | HSA | FIX | 49 |
| FIX | QSFNDFTRVVGGE | HSA | FIX | 108 |
| FIX | QSFNDFTRTVGGED | HSA | FIX | 50 |
| FIX | QSFNDFTRLVGGED | HSA | FIX | 51 |
| FIX | QSFNDFTRGVGGED | HSA | FIX | 52 |
| FIX | QSFNDFTRVVGGED NGS | HSA | FIX | 53 |
| FIX | QSFNDFTRVVGGEDN | HSA | FIX | 54 |
| FIX | PERGDNNLTRIVIGGQE GS | HSA | FX | 109 |
| FIX | PERGDNNLTRIVGGQE | HSA | FX | 61 |
| FIX | PERGDNNLTRIVGGQ | HSA | FX | 110 |
| FIX | DNNLTRIVGGQ | HSA | FX | 111 |
| FIX | SVSQTSKLTRAETVFPDVD | Fc | FIX | 62 |
| FIX | QSFNDFTRVVGGED N | Fc | FIX | 63 |
| FIX (1-412) | SVSQTSKLTRAETVFPDVD | Fc | FIX | 64 |
| FIX | ASKPQGR IVGG | HSAdelDAH | FVII | 112 |
| FIX | KRNASKPQGRIVGGKV | HSA | FVII | 65 |
| FIX | PEEPQLR MKNNEEAED | HSA | FVIII | 66 |
| FIX | DNSPSFIQIR SVAKKHPKT | HSA | FVIII | 67 |
| FIX | LSKNNAIEPRSFSQNSRHPS | HSA | FVIII | 68 |
| FIX | DEDENQSPRSFQKKTRHYFIA | HSA | FVIII | 69 |
| FIX | SPHVLRNRAQSGSVPQ | HSA | FVIII | 70 |
| FVII or FVIIa | PEEPQLRMKNNEEAEDYDDDLTDS | HSA | FVIII | 71 |
| FVII or FVIIa | DDDNSPSFIQIR SVAKKHPKTWVHYAAEEED | HSA | FVIII | 72 |
| FVII or FVIIa | LSKNNAIEPRSFSQNSRHPSTRQKQFNA | HSA | FVIII | 73 |
| FVII or FVIIa | DEDENQSPR SFQKKTRHYFIAA | HSA | FVIII | 74 |
| FVII or FVIIa | DYGMSSSPHVLRNRAQSGSVPQFKKVVFQEFT | HSA | FVIII | 75 |
| FVIII | Derived from cleavage sites of | HSA | FVIII, FIX or Fgn | |
| | FVIII, FIX or Fibrinogen | | | |
| VWF | Derived from cleavage sites of | HSA | FIX, FVIII, VWF | |
| | VWF, FVIII, or FIX | | | |
| VWF | DIYDEDENQSPRSFOKKTRHYFIA | HSA | FVIII | 76 |
| VWF | DNSPSFIQIR SVAKKHP | HSA | FVIII | 77 |
| VWF | LSKNNAIEPR SFSQNSRHPS | HSA | FVIII | 78 |

In the case of linkers derived from the N-terminal region of the FIX activation peptide, according to the natural polymorphism T148-A148 the sequences may also contain A instead of T at this position.

TABLE 3b
Examples of possible constructs with two or more cleavage sites

| | | | | |
|---|---|---|---|---|
| | | | Linker derived | SEQ |
| Coagulation | | | from (partially | ID |
| factor | Linker | HLEP | incl. Modifications) | NO: |

Linker with two cleavage sites

| | | | | |
|---|---|---|---|---|
| FIX | SVSQTSKLTR AETVFPDVTQPERGDNNLTRIVGGQE | HSA | FIX, FX | 79 |
| FIX | SKLTR AETVFPDNNLTRIVGGQE | HSA | FIX, FX | 80 |
| FIX | R AETVFPDV TQPERGDNNLTRIVGGQE | HSA | FIX, FX | 81 |
| FIX | R AETVFPERGDNNLTRIVVGGQE | HSA | FIX, FX | 82 |
| FIX | SVSQTSKLTR AETVFPDVDYV NNLTRIVGGQE | HSA | FIX, FX | 83 |
| FIX | SVSQTSKLTR AETVFPDVD NNLTRIVGGQE | HSA | FIX, FX | 84 |
| FIX | SVSQTSKLTR AETVFPDVD NNLTRIVGGQE | HSA | FIX, FX | 85 |
| FIX | SVSQTSKLTRAETVFPDVDYVNSTEAETILDN | HSA | FIX | 86 |
| | ITQSTQSFNDFTRVVGGEDA | | | |
| FIX | SVSQTSKLTR AETVFPDVQSFNDFTRVVGGED | HSA | FIX | 87 |
| FIX | SVSQTSKLTR AETVFPDVDSFNDFTRVVGGED | HSA | FIX | 88 |
| FIX | SVSQTSKLTR AETVFPDVNASKPQGRIVGGKV | HSA | FIX and FVII | 89 |
| FIX | SVSQTSKLTR AETVFPDVNASKPQGRLVGGKV | HSA | FIX and FVII | 90 |
| FIX | SVSQTSKLTR AETVFPDVNASKPQGRTVGGKV | HSA | FIX and FVII | 91 |
| FIX | SVSQTSKLTR AETVFPDVD | Fc | | 92 |

Variants and fragments of the linkers described in tables 3a and 3b are also encompassed in the present invention as long as the linkers can still be cleaved by the protease or the proteases that cleave the linkers of tables 3a and 3b or by the type of proteases defined above. The term “variants” includes insertions, deletions and substitutions, either conservative or non-conservative, and other chemical modifications.

Other combinations of the cleavage sequences described above and their variants shall be included in the present invention.

In another embodiment, amino acid substitutions are included that change the post-translational modification pattern of the peptide linker. These can be, for example, substitutions of amino acids that are glycosylated, sulphated, or phosphorylated.

In another embodiment of the invention the peptide linker between the therapeutic polypeptide and the HLEP moiety contains consensus sites for the addition of posttranslational modifications. Such modifications may comprise glycosylation sites. For example, such modifications may comprise at least one N-glycosylation site of the structure Asn-X-Ser/Thr, wherein X denotes any amino acid except proline. Furthermore, such N-glycosylation sites may be inserted close to the amino and/or carboxy terminus of the peptide linker such that they are capable of shielding potential neo-epitopes which might develop at the sequences where the therapeutic polypeptide moiety is transitioning into the peptide linker or where the peptide linker is transitioning into, for example, an albumin moiety sequence.

### BRIEF DESCRIPTION OF THE FIGURES

FIG. 1: In vitro activation of FIX-albumin fusion proteins by FXIa at 37° C. at a molar ratio of FXIa to fusion protein of about 1:500. One fusion protein with non-cleavable linker (1478/797) and two fusion proteins with cleavable linker (1088/797 and 1089/797) were used. Samples were analyzed by SDS-PAGE under reducing conditions followed by Coomassie blue staining

FIG. 2: Pharmacokinetics of activated rec FIX and FIX-albumin fusion proteins with and without cleavable linker in comparison to non-activated fusion proteins.

FIG. 3: Inactivation of activated rec FIX or FIX-albumin fusion protein by AT. Residual FIX activity was determined after 120 min using a non-activated partial thromboplastin time assay.

### EXAMPLES

### Example 1

### Generation of cDNAs Encoding FIX and FIX-Albumin Fusion Proteins

Factor IX coding sequence was amplified by PCR from a human liver cDNA library (ProQuest, Invitrogen) using primers We1403 and We1404 (SEQ ID NOS:5 and 6). After a second round of PCR using primers We1405 and We1406 (SEQ ID NOS:7 and 8) the resulting fragment was cloned into pCR4TOPO (Invitrogen). From there the FIX cDNA was transferred as an EcoRI Fragment into the EcoRI site of pIRESpuro3 (BD Biosciences) wherein an internal XhoI site had been deleted previously. The resulting plasmid was designated pFIX-496 and was the expression vector for factor IX wild-type.

For the generation of albumin fusion constructs the FIX cDNA was reamplified by PCR under standard conditions using primers We2610 and We2611 (SEQ ID NOS:9 and 10) deleting the stop codon and introducing an XhoI site instead. The resulting FIX fragment was digested with restriction endonucleases EcoRI and XhoI and ligated into an EcoRI/BamH1 digested pIRESpuro3 together with one XhoI/BamH1 digested linker fragment as described below.

Two different glycine/serine linker fragments without internal cleavage sites were generated: Oligonucleotides We2148 and We2150 (SEQ ID NOS:11 and 12) were annealed in equimolar concentrations (10 pmol) under standard PCR conditions, filled up and amplified using a PCR protocol of a 2 min. initial denaturation at 94° C. followed by 7 cycles of 15 sec. of denaturation at 94° C., 15 sec. of annealing at 55° C. and 15 sec. of elongation at 72° C., and finalized by an extension step of 5 min at 72° C. The same procedure was performed using oligonucleotides We2156 and We2157 (SEQ ID NOS:13 and 14). The resulting linker fragments were digested with restriction endonucleases XhoI and BamH1 and used separately in the above described ligation reaction. The resulting plasmids therefore contained the coding sequence for FIX and a C-terminal extension of a glycine/serine linker.

Two different cleavable linker fragments derived from the activation sites of FIX were generated: Oligonucleotides We2335 and We2336 (SEQ ID NOS:15 and 16), containing the activation cleavage site of the FIX light chain/activation peptide border region, were annealed, filled, and amplified as described above. The resulting linker fragment was digested with restriction endonucleases XhoI and BamH1 and used in the above described ligation reaction. The resulting plasmid therefore contained the coding sequence for FIX and a C-terminal extension of a cleavable FIX sequence (amino acids 136 to 154 of SEQ ID NO:2). In a subsequent site directed mutagenesis reaction with a commercially available mutagenesis kit (QuickChange XL Site Directed Mutagenesis Kit, Stratagene) using oligonucleotides We2636 and We2637 (SEQ ID NOS:17 and 18) the XhoI site was deleted.

For generation of the second cleavable linker fragment derived from FIX, the same procedure was performed using oligonucleotides We2337 and We2338 (SEQ ID NOS:19 and 20) for linker construction. The resulting linker fragment was digested with restriction endonucleases XhoI and BamH1 and used in the above described ligation reaction. The resulting plasmid now contained the coding sequence for FIX and a C-terminal extension of a cleavable FIX sequence derived from the activation cleavage site of the FIX activation peptide/heavy chain border region (amino acids 173 to 186 of SEQ ID NO:2). Oligonucleotides We2638 and We 2639 (SEQ ID NOS:21 and 22) were used for deletion of the XhoI site as described above.

In the next cloning step the above generated plasmids were digested with BamH1 and a BamH1 fragment containing the cDNA of mature human albumin was inserted. This fragment had been generated by PCR on an albumin cDNA sequence using primers We1862 and We1902 (SEQ ID NOS:23 and 24) under standard conditions.

The final plasmids with non-cleavable glycine/serine linkers were designated pFIX-980 (SEQ ID NO:30) and pFIX-986 (SEQ ID NO:31), respectively. The final plasmids with cleavable linkers derived from FIX sequences were designated pFIX-1088 (SEQ ID NO:40) and pFIX-1089 (SEQ ID NO:49), respectively. Their linker sequences and the C-terminal FIX and N-terminal albumin sequences are outlined below. Proteolytic cleavage sites within the linkers are indicated with arrows, the FIX derived linker sequences are underlined.

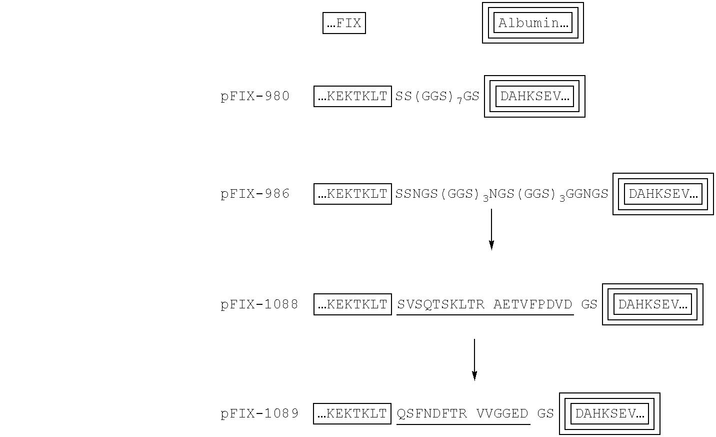

For expression in CHO cells the coding sequences for the FIX albumin fusion protein were transferred into vectors pIRESneo3 (BD Biosciences) or pcDNA3.1 (Invitrogen), respectively.

For efficient processing of the propeptide in cells expressing FIX in high amounts coexpression of furin is required (Wasley L C et al. 1993. PACE/Furin can process the vitamin K-dependent pro-factor IX precursor within the secretory pathway. J. Biol. Chem. 268:8458-8465). Furin was amplified from a liver cDNA library (Ambion) using primers We1791 and We1792 (SEQ ID NOS:25 and 26). A second round of PCR using primers We1808 and We1809 (SEQ ID NOS:27 and 28) yielded a furin fragment where the carboxyterminal transmembrane domain (TM) was deleted and a stop codon introduced; this fragment was cloned into pCR4TOPO (Invitrogen). From there the furinΔTM cDNA was transferred as an EcoRI/NotI Fragment into the EcoRI/NotI sites of pIRESpuro3 (BD Biosciences) wherein an internal XhoI site had been deleted previously. The resulting plasmid was designated pFu-797. This plasmid was cotransfected with all FIX constructs in a 1:5 (pFu-797: pFIX-xxx) molar ratio. The amino acid sequence of the secreted furin encoded by pFu-797 is given as SEQ-ID NO:29.

### Example 2

### Transfection and Expression of FIX and FIX-Albumin Fusion Proteins

Plasmids were grown up in E. coli TOP10 (Invitrogen) and purified using standard protocols (Qiagen). HEK-293 cells were transfected using the Lipofectamine 2000 reagent (Invitrogen) and grown up in serum-free medium (Invitrogen 293 Express) in the presence of 50 ng/ml Vitamin K and 4 μg/ml Puromycin. Transfected cell populations were spread through T-flasks into roller bottles or small-scale fermenters from which supernatants were harvested for purification.

Alternatively, CHO K1 or DG44 cells (Invitrogen) were transfected using the Lipofectamine 2000 reagent (Invitrogen) and grown up in serum-free medium (Invitrogen CD-CHO) in the presence of 50 ng/ml Vitamin K and 500-750 ng/ml Geneticin. High expressing clones were selected and spread through T-flasks into roller bottles or small-scale fermenters from which supernatants were harvested for purification.

### Example 3

### Purification of FIX and FIX-Albumin Fusion Proteins

Cell culture harvest containing FIX or FIX albumin fusion protein was applied on a Q-Sepharose FF column previously equilibrated with 50 mM TrisxHCl/100 mM NaCl buffer pH 8.0. Subsequently, the column was washed with equilibration buffer containing 200 mM NaCl. Elution of the bound FIX or FIX fusion protein was achieved by a salt gradient using 50 mM TrisxHCl/200 mM NaCl buffer pH 8.0 as a basis. The eluate was further purified by column chromatography on a hydroxylapatite resin. For this purpose, the eluate of the Q-Sepharose FF column was loaded on a hydroxylapatite chromatography column equilibrated with 50 mM TrisxHCl/100 mM NaCl buffer pH 7.2. The column was washed with the same buffer and FIX or FIX-HSA were eluted using a potassium phosphate gradient at pH 7.2. The eluate was dialyzed to reduce the salt concentration and used for biochemical analysis as well as for determination of the pharmacokinetic parameters. FIX antigen and activity were determined as described in example 5.

### Example 4

### Alternative Purification Scheme of FIX and FIX-Albumin Fusion Proteins

As described in example 3, cell culture harvest containing FIX or FIX albumin fusion protein was purified by chromatography on Q-Sepharose FF. The Q-Sepharose eluate was further purified by chromatography on a Heparin-Fractogel column. For this purpose, the Heparin-Fractogel column was equilibrated using 50 mM Tris×HCl, 50 mM NaCl pH 8.0 buffer (EP), the Q-Sepharose FF eluate was applied and the column was washed with equilibration buffer containing 75 mM NaCl. FIX or FIX albumin fusion protein, respectively, was eluted using EP adjusted to 300 mM NaCl. The Heparin-Fractogel eluate was further purified by chromatography on a hydroxylapatite chromatography column as described in example 3. The purified FIX resp. FIX albumin fusion protein concentrate was subjected to FIX activity and antigen determination according to example 5 and characterized by further in vitro and in vivo investigations.

### Example 5

### Determination of FIX Activity and Antigen

FIX activity was determined as clotting or coagulation activity (FIX:C) using commercially available aPTT reagents (Pathromtin SL and FIX depleted plasma, Dade Behring). An internal substandard calibrated against the WHO International FIX concentrate Standard (96/854) was used as a reference.

FIX antigen (FIX:Ag) was determined by an ELISA acc. to standard protocols known to those skilled in the art. Briefly, microtiter plates were incubated with 100 μL per well of the capture antibody (Paired antibodies for FIX ELISA 1:200, Cedarlane, but other sources of appropriate antibodies may also be applied) overnight at ambient temperature. After washing plates three times with washing buffer B (Sigma P3563), each well was incubated with 200 μL blocking buffer C (Sigma P3688) for one hour at ambient temperature. After another three wash steps with buffer B, serial dilutions of the test sample in buffer B as well as serial dilutions of a substandard (SHP) in buffer B (volumes per well: 100 μL) were incubated for two hours at ambient temperature. After three wash steps with buffer B, 100 μL of a 1:200 dilution of the detection antibody (Paired antibodies for FIX ELISA, peroxidase labelled, Cedarlane) in buffer B were added to each well and incubated for another two hours at ambient temperature. After three wash steps with buffer B, 100 μL of substrate solution (TMB, Dade Behring, OUVF) were added per well and incubated for 30 minutes at ambient temperature in the dark. Addition of 100 μL undiluted stop solution (Dade Behring, OSFA) prepared the samples for reading in a suitable microplate reader at 450 nm wavelength. Concentrations of test samples were then calculated using the standard curve with standard human plasma as reference.

### Example 6

### Comparison of FIX-Activity/FIX-Antigen Ratio of Different FIX-Albumin Fusion Proteins in Cell Culture Supernatant

Cell culture supernatants of HEK cells transfected with DNA constructs coding for FIX-albumin fusion proteins that contained different linker peptides were subjected to FIX activity and antigen testing as described above (see example 5). The ratio of FIX:C to FIX:Ag was calculated representing a measure directly proportional to molar specific activity of the different constructs.

The results shown in table 4 indicate that there is an increase in activity/antigen ratio upon introduction of cleavable linkers into the FIX-HSA molecule. It also shows that the cleavable linker peptide should have a length of more than two amino acids in order to provide clearly increased activity/antigen ratios.

TABLE 4

FIX:C/FIX:Ag ratios of FIX-albumin fusion proteins
containing different linker peptides

| | | | |
|---|---|---|---|
| | | | Fold increase |
| | | | compared to |
| | | | fusion protein |
| | | | 980/797 with |
| | | | non-cleavable |
| FIX-HSA | | | linker |
| construct | Linker | FIX:C/FIX:Ag | (GGGGGGV) |

| | | | |
|---|---|---|---|
| 1182/797 | None | <0.031 | |
| 1366/797 | RI | <0.068 | |
| 1478/863 | GGGGGGV | 0.041 | — |
| | (Sheffield et al.) | | |
| 980/797 | SS(GGS)hd 7GS | 0.070 | 1.7 |
| 986/797 | SSNGS(GGS)3NGS | 0.076 | 1.9 |
| | (GGS)3GGNGS | | |
| 1483/863 | SVSQTSKLTR AETVFPDVD | 0.688 | 16.8 |
| | GSGGS | | |
| 1088/797 | SVSQTSKLTR AETVFPDVD GS | 0.832 | 20.3 |
| 1365/797 | SVSQTSKLTR AETVFPDVD | 0.630 | 15.4 |
| 1482/863 | SVSQTSKLTR AETVFP | 0.482 | 11.8 |
| 1087/797 | SVSQTSKLTR AETVFPDVD GS | 0.472 | 11.5 |
| | (FIX deltaKLT) | | |
| 1089/797 | QSFNDFTR VVGGED GS | 0.532 | 13.0 |
| 1091/797 | PERGDNNLTR IVGGQE GS | 0.111 | 2.7 |

### Example 7

### Comparison of FIX and FIX-Albumin Fusion Proteins in respect to Molar Specific Activity, Terminal In Vivo Half-Life and In Vivo Recovery in Rats or Rabbits

Purified recombinant wild type FIX (rFIX 496/797) and FIX-albumin fusion proteins (rFIX 980/797, rFIX 986/797, rFIX-1088/797 and rFIX 1089/797) were tested for FIX activity in a clotting assay as described above. In parallel, the difference of the optical density at 280 and 320 nm was determined as a measure for protein concentration (OD280-320). The ratios of activity per OD280-320 were calculated and based on the molar optical densities the molar specific activities were calculated. In the following table 5 the results are summarized.

TABLE 5
Molar specific activities of wt FIX compared to FIX-albumin fusions

| | Optical | FIX | Activity/ | Molar |
|---|---|---|---|---|
| | density | clotting | Vol/OD | specific |
| | (OD280- | activity | (IU/mL/ | activity* |
| Linker | 320) | (IU/mL) | OD) | (IU/nmol) |

| | | | | | |
|---|---|---|---|---|---|
| rFIX,wt (496/797 | | 0.3798 | 21.2 | 55.8 | 4, 23 |
| rFIX-HSA (non-cleavable, 1478/863 [symbol] | GGGGGGV (Sheffield et al.) | 2.9189 | 5.8 | 2.0 | 0, 23 |
| rFIX-HSA (non-cleavable, 980/797) | SS (GGS)₇ GS | 1.1122 | 3.4 | 3.0 | 0, 36 |
| rFIX-HSA non-cleavable, 986/797) | [symbol] SS NGS (GGS)3NGS (GGS)3 GGN GS [symbol] | 0.8107 | 3.2 | 4.0 | 0, 45 |
| rFIX-HSA (cleavable, 1088/797) | FXIa cleavable | 0.3421 | 11.9 | 34.8 | 3, 95 |
| rFIX-HSA (cleavable, 1089/797) | FXIa cleavable | 0.4512 | 11.3 | 25.0 | 2, 84 |

*Molar specific activity based on activity, optical density and the following molar optical densities:
Molar optical density of FIX: OD (280 nm, 1 mol/L) = 75 810
Molar optical density of albumin: OD (280 nm, 1 mol/L) = 37 791
Molar optical density of FIX-albumin fusion protein: OD (280 nm, 1 mol/L) = 113 601 [symbol]
indicates data missing or illegible when filed

Taking the results summarized in Table 5 into account, two constructs that were generated according to the present invention show highly increased molar specific activities compared to the fusion proteins with non-cleavable linkers. In addition, the molar specific activity of these constructs was only moderately decreased compared to wild type rFIX.

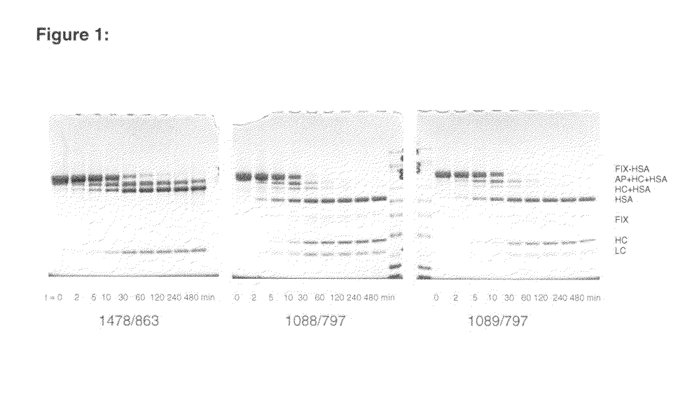

In vitro investigations of the proteolytic cleavage reactions by Factor XIa (FXIa) confirmed that FIX-albumin fusion proteins containing a cleavable linker such as construct no. 1088/797 or 1089/797 are activated and in parallel the linker is cleaved resulting in release of the albumin moiety (FIG. 1). The fusion protein with non-cleavable linker did not show a corresponding release of the albumin moiety.

In the case of FVIIa as cleaving protease in the presence of tissue factor, the FIX-albumin fusion proteins 1088/797 or 1089/797 containing a cleavable linker also showed release of the albumin moiety in parallel to release of the FIX activation peptide (Data not shown).

In addition to determination of molar specific coagulation activity, the polypeptides no. 496/797, 980/797, 986/797, 1088/797 and 1089/797 described above were administered intravenously to narcotized CD/Lewis rats (6 rats per substance) and/or rabbits (4 rabbits per substance) with a dose of 50 IU/kg body weight. Blood samples were drawn prior to test substance administration and at appropriate intervals starting at 5 minutes after administration of the test substances. FIX antigen content was subsequently quantified by an ELISA assay specific for human Factor IX (see above). The mean values of the respective groups were used to calculate in vivo recovery after 5 min. Half-lives for each protein were calculated using the time points of the beta phase of elimination (terminal half-life) according to the formula t_{1/2}=ln 2/k, whereas k is the slope of the regression line obtained upon plotting FIX:Ag levels in logarithmic scale and time in linear scale.

Calculated in vivo half-lives are summarized in table 6. In rats as well as in rabbits the in vivo half-lives of the FIX-albumin fusion proteins were found to be significantly increased in comparison to non-fused wild-type recombinant FIX prepared in-house or in comparison to the commercially available recombinant FIX product BeneFIX®. The in vivo half-lives of the albumin fusion proteins compared to BeneFIX® were increased to about 200-400%, depending on the animal species or construct used (Table 6).

To evaluate the in vivo recovery, the FIX antigen levels measured per mL of plasma at their maximum concentrations after intravenous administration (t=5 min) were related to the amount of product applied per kg. Alternatively, a percentage was calculated by relating the determined antigen level (IU/mL) 5 min post infusion to the theoretical product level expected at 100% recovery (product applied per kg divided by an assumed plasma volume of 40 mL per kg). The in vivo recoveries (IVR) of the FIX-albumin fusion proteins were significantly higher than the in vitro recoveries of rFIX (496/797) or BeneFIX® (Table 7).

TABLE 6
Terminal in vivo half-lives of FIX preparations derived from recombinant
expression (BeneFIX ®, rFIX 496/797) and FIX albumin fusion
proteins (rFIX 980/797, rFIX 986/797, rFIX 1088/797, and rFIX
1089/797) after intravenous administration of 50 IU/kg into rats
and/or 50 IU/kg into rabbits, respectively.

| | Rat experiments | | | |
|---|---|---|---|---|
| | PSR18-05, PSR06-05, | | Rabbit experiment | |
| | PSR02-05 | | PSK11-05 | |
| | Terminal | | Terminal | |
| | half- | relative to | half- | relative to |
| | life (h) | BeneFIX [%] | life (h) | BeneFIX [%] |
| rFIX 496/797 | 4.5* | 91 | n.t. | n.t. |
| rFIX 980/797 | 11.6* | 234 | 36.9° | 410 |
| | | | 29.3° | 326 |
| | | | (2^{nd} exp.) | |
| rFIX 986/797 | 10.5* | 212 | n.t. | n.t. |
| rFIX 1088/797 | 8.3* | 168 | 30.3° | 337 |
| rFIX 1089/797 | 10.5* | 212 | n.t. | n.t. |
| BeneFIX | 4.95* | 100 | 9.0° | 100 |
| | (mean of | | | |
| | 5.3 and | | | |
| | 4.6) | | | |

*Determined between 120 and 1440 min
°Determined between 4 and 96 h

TABLE 7
In vivo recoveries (amount of substance 5 minutes post administration)
of recombinant FIX preparations (BeneFIX, rFIX 496/797) and FIX
albumin fusion proteins (rFIX 1088/797, rFIX 1089/797) after intravenous
administration of 50 IU/kg into rats. The percentage of in vivo recovery
was calculated based on an assumed plasma volume of 40 mL/kg.

rat experiment

| | in vivo recovery | relative to |
|---|---|---|
| | IU/dL per IU/kg/[%]* | BeneFIX [%] |
| rFIX | 0.462/18.5 | 74.6 |
| 496/797 | | |
| rFIX | 1.034/41.4 | 166.5 |
| 1088/797 | | |
| rFIX | 1.063/42.5 | 171.2 |
| 1089/797 | | |
| BeneFIX | 0.621/24.8 | 100 |

*Calculated based on a plasma volume of 40 mL/kg

### Example 8

### In Vitro Activation of FIX Albumin Fusion Proteins with/without Cleavable Linker (1088/797 and 980/797) and Determination of Pharmacokinetics in Rats

FIX-albumin fusion proteins and rec FIX were activated in vitro using commercially available Factor XIa (Kordia). Briefly, identical molar amounts of FIX or FIX-albumin fusion protein (3.0×10⁻⁶ mol/L) were activated at 37° C. in solution in the presence of FXIa (1.9×10⁻⁸ mol/L) and CaCl₂ (1.5 mmol/L) buffered at pH 6.8. After complete activation as shown by SDS-PAGE the reaction was stopped by addition of a 5×molar excess of C1-Inhibitor (Berinert® P) based on the amount of FXIa. The samples were stored frozen below −70° C. until start of pharmacokinetic investigation.

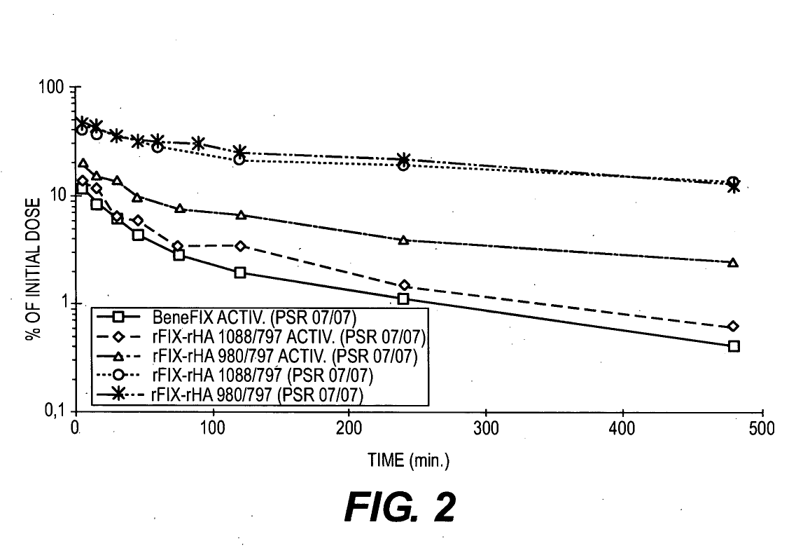

A pharmacokinetic investigation of the activated FIX and the FIX-albumin fusion proteins was performed in rats as described in example 7 and the results were compared to a pharmacokinetic results covering non-activated fusion proteins. It turned out that the activated fusion proteins demonstrated significantly reduced half-lives as well as AUC's compared to the non-activated molecules (FIG. 2). Upon activation the FIX-fusion protein with cleavable linker (1088/797) showed a pharmacokinetic behaviour very similar to activated rec FIX (BeneFIX®) whereas the activated fusion protein with non-cleavable linker (980/797) resulted in a higher initial as well as terminal half-life compared to activated fusion protein 1088/797 with cleavable linker. Therefore, in this example, the cleavable linker results in increased elimination of the coagulation factor after activation and, therefore, avoids accumulation of potentially thrombogenic, activated fusion proteins with extended half-lives.

### Example 9

### Comparison of FIX-Albumin Fusion Proteins with/without Cleavable Linker in Respect to Inactivation Rate of the Activated Coagulation Factors by Antithrombin III (AT)

FIX fusion proteins with (1088/797) and without (980/797) cleavable linker were activated by incubation with FXIa as described in example 8. The activated factors were incubated with AT for 120 min and residual FIXa activity was determined using a manual FIX clotting assay method without activation (naPTT, see below) acc. to Schnitger and Gross. As control samples the activated FIX-albumin fusion proteins were used in presence of the same amount of AT but without incubation.

The FIX activity was determined with the aid of a non-activated partial thromboplastin time assay (naPTT) using FIX deficient plasma from Dade Behring. The samples were prediluted in a buffer of pH 6.8 containing His, Gly, Sucrose, and Tween® 80. The whole determination was performed using coagulometers acc. to Schnitger & Gross. A mixture of 0.1 ml F IX deficient plasma, 0.1 ml sample, and 0.1 ml of 0.1% Phospholipids (Rhone-Poulenc-Nattermann, 1:3 prediluted in imidazole buffer supplemented with 1% HSA) was incubated for 2 minutes at 37° C. The coagulation reaction was initiated by adding 0.1 ml 0.025 mol/l CaCl₂ solution and the clotting time was determined.

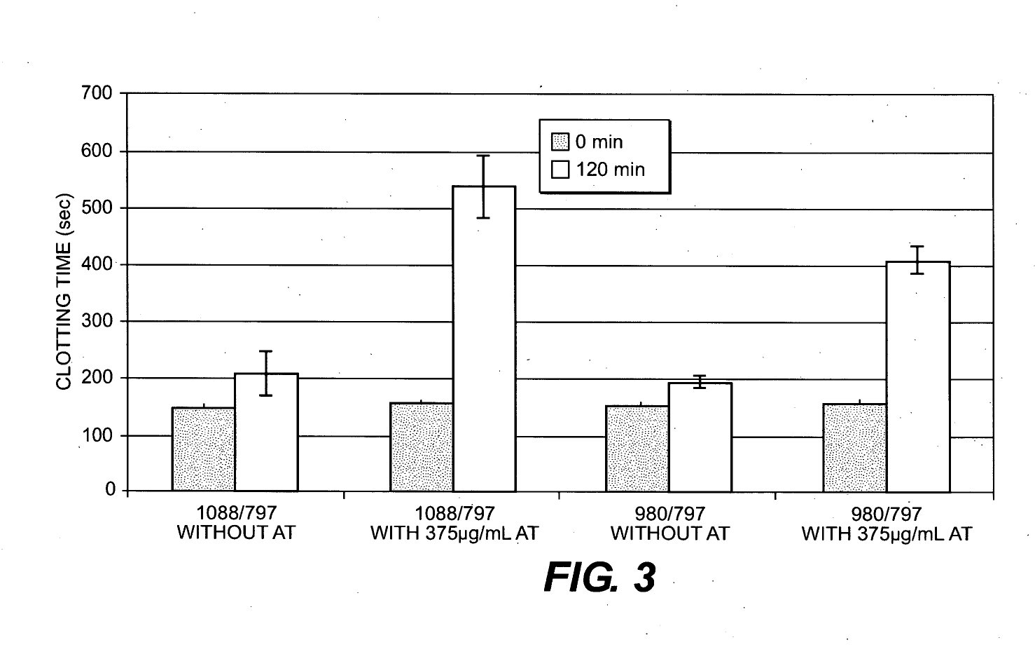

FIG. 3 shows the results of a corresponding inactivation experiment. In the case of the fusion protein with cleavable linker (1088/797) an increase in clotting time from 210 to 540 sec (factor of 2.57×) demonstrated an accelerated inactivation process of FIXa activity by AT compared to a fusion protein with non-cleavable linker (980/797) that only showed an increase from 196 to 411 sec (factor of 2.10 x). Most probably, the albumin residue sterically affects the AT dependent inactivation process in the case of the fusion protein with non-cleavable linker whereas in the case of the fusion protein with cleavable linker the albumin residue is cleaved off resulting in an accelerated inactivation by AT.

FIG. 1: In vitro activation of FIX-albumin fusion proteins by FXIa at 37° C. at a molar ratio of FXIa to fusion protein of about 1:500. One fusion protein with non-cleavable linker (1478/797) and two fusion proteins with cleavable linker (1088/797 and 1089/797) were used. Samples were analyzed by SDS-PAGE under reducing conditions followed by Coomassie blue staining.

FIG. 2: Pharmacokinetics of activated rec FIX and FIX-albumin fusion proteins with and without cleavable linker in comparison to non-activated fusion proteins.

FIG. 3: Inactivation of activated rec FIX or FIX-albumin fusion protein by AT. Residual FIX activity was determined after 120 min using a non-activated partial thromboplastin time assay.

1. A fusion protein comprising:
a) a coagulation factor,
b) a half-life enhancing polypeptide (HLEP) chosen from albumin and an immunoglobulin without an antigen binding domain, and
c) a peptide linker which joins the coagulation factor and the half-life enhancing polypeptide;
wherein the peptide linker is cleavable by proteases involved in coagulation or is activated by coagulation enzymes, and wherein the fusion protein has, in comparison to a respective fusion protein linked by a non-cleavable linker having the amino acid sequence GGGGGGV (SEQ ID NO: 94), at least one of the following properties:
i) an increased molar specific activity in at least one coagulation-related assay,
ii) an increased inactivation rate of the activated coagulation factor after the peptide linker is proteolytically cleaved in a coagulation-related mode, and
iii) an increased elimination rate of the activated coagulation factor after the peptide linker is proteolytically cleaved in a coagulation-related mode.

2. The fusion protein according to claim 1 wherein said fusion protein has, in comparison to the respective fusion protein linked by a non-cleavable linker having the amino acid sequence GGGGGGV (SEQ ID NO: 94), a higher in vivo recovery.

3. The fusion protein according to claim 1 wherein said fusion protein has an increased half-life in plasma compared to the respective fusion protein linked by a non-cleavable linker having the amino acid sequence GGGGGGV (SEQ ID NO: 94).

4. The fusion protein according to claim 1 wherein the coagulation factor is a vitamin-K dependent coagulation factor.

5. The fusion protein according to claim 4 wherein the vitamin-K dependent coagulation factor is FVIIa or FIX.

6. The fusion protein according to claim 1 wherein the half-life enhancing polypeptide is an immunoglobulin without an antigen binding domain.

7. The fusion protein according to claim 1 wherein the peptide linker is cleavable by FXIa and/or FVIIa/TF.

8. The fusion protein according to claim 1 wherein the molar specific coagulation-related activity of the fusion protein is increased at least 25% compared to that of the respective fusion protein linked by a non-cleavable linker having the amino acid sequence GGGGGGV (SEQ ID NO: 94) in at least one coagulation-related assay.

9. The fusion protein according to claim 1 wherein the inactivation rate of the coagulation factor after cleavage of the peptide linker is increased by at least 10% as compared to the inactivation rate of the coagulation factor in a corresponding fusion protein linked by a non-cleavable linker having the amino acid sequence GGGGGGV (SEQ ID NO: 94).

10. The fusion protein according to claim 1 wherein the elimination rate of the coagulation factor after cleavage of the peptide linker is increased by at least 10% as compared to the elimination rate of the coagulation factor in a corresponding fusion protein linked by a non-cleavable linker having the amino acid sequence GGGGGGV (SEQ ID NO: 94).

11. The fusion protein according to claim 1 wherein the linker is cleavable by one or more proteases that activate the coagulation factor in vivo.

12. The fusion protein according to claim 11, wherein the rate of the linker cleavage is not delayed by more than a factor of 3 compared to the rate of the activation of said coagulation factor.

13. The fusion protein according to claim 1 wherein the linker is cleavable by the protease or proteases that are activated upon involvement of the coagulation factor.

14. The fusion protein according to claim 1 wherein the linker is cleavable by one or both of FXIa and FVIIa/TF and wherein the coagulation factor is FIX.

15. The fusion protein according to claim 1 wherein the linker is cleavable by one or both of FXa and FVIIa/TF and wherein the coagulation factor is FVIIa.

16. The fusion protein according to claim 1 wherein the linker comprises a sequence chosen from SEQ ID NOS:36, 37, 38, 95, 39, 40, 96, 97, 98, 99, 100, 101, 102, 103, 104, 105, 41, 42, 43, 44, 45, 46, 106, 107, 47, 48, 49, 108, 50, 51, 52, 53, 54, 109, 61, 110, 111, 62, 63, 64, 112, 65, 66, 67, 68, 69, 70, 71, 72, 73, 74, 75, 76, 77, 78, 79, 80, 81, 82, 83, 84, 85, 86, 87, 88, 89, 90, 91, and 92.

17. A polynucleotide comprising a polynucleotide sequence encoding the fusion protein according to any one of claim 1.

18. A plasmid or vector comprising the polynucleotide according to claim 17.

19. The plasmid or vector according to claim 18, which is an expression vector.

20. The plasmid or vector according to claim 18, wherein the vector is a transfer vector for use in human gene therapy.

21. A host cell comprising the polynucleotide according to claim 17.

22. A method of producing the fusion protein according to any one of claim 1, comprising culturing host cells comprising a polynucleotide encoding said fusion protein under conditions such that the fusion protein is expressed by the host cells.

23. The method of claim 22 further comprising recovering the fusion protein from the host cells or from the culture medium.

24. A pharmaceutical composition comprising the fusion protein according to claim 1, or comprising a polynucleotide encoding said fusion protein or a plasmid or vector comprising a polynucleotide encoding said polynucleotide.

25. A method of administering an effective amount of the fusion protein according to claim 1 to a patient in need thereof, comprising administering said fusion protein, or comprising administering a composition comprising a polynucleotide encoding said fusion protein via a gene therapy protocol.

26. The method according to claim 25, wherein the patient suffers from a blood coagulation disorder.

27. The method according to claim 26, wherein the blood coagulation disorder is hemophilia B.

28. The method according to claim 26, wherein the blood coagulation disorder is FVII and/or FVIIa deficiency.

29. The method according to claim 26, wherein the blood coagulation disorder is hemophilia A.

30. The method according to claim 26, wherein the administration comprises administering a composition comprising a polynucleotide encoding said fusion protein fusion protein via a gene therapy protocol.

31. The method according to claim 25, wherein the fusion protein is effective to act in the patient as a procoagulant.


SEQUENCE LISTING (117 sequences)

SEQ ID NO: 1 - protein, 585 aa, Homo sapiens [sequence omitted]

SEQ ID NO: 2 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 3 - DNA, 1386 bp, Homo sapiens
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 4 - DNA, 1830 bp, Homo sapiens
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 5 - DNA, 21 bp, Artificial Sequence
  Description of Artificial Sequence Synthetic oligonucleotide
ccactttcacaatctgctagc

SEQ ID NO: 6 - DNA, 23 bp, Artificial Sequence
  Description of Artificial Sequence Synthetic oligonucleotide
caattccaatgaattaaccttgg

SEQ ID NO: 7 - DNA, 21 bp, Artificial Sequence
  Description of Artificial Sequence Synthetic oligonucleotide
atgcagcgcgtgaacatgatc

SEQ ID NO: 8 - DNA, 25 bp, Artificial Sequence
  Description of Artificial Sequence Synthetic oligonucleotide
tcattaagtgagctttgttttttcc

SEQ ID NO: 9 - DNA, 21 bp, Artificial Sequence
  Description of Artificial Sequence Synthetic oligonucleotide
gattcgaattcgcccttatgc

SEQ ID NO: 10 - DNA, 32 bp, Artificial Sequence
  Description of Artificial Sequence Synthetic oligonucleotide
cgctcgaggtgagctttgttttttccttaatc

SEQ ID NO: 11 - DNA, 52 bp, Artificial Sequence
  Description of Artificial Sequence Synthetic oligonucleotide
ctcgagcgggggatctggcgggtctggaggctctggagggtcgggaggctct

SEQ ID NO: 12 - DNA, 46 bp, Artificial Sequence
  Description of Artificial Sequence Synthetic oligonucleotide
ggatccagatcccccagagcctccagagcctcccgaccctccagag

SEQ ID NO: 13 - DNA, 56 bp, Artificial Sequence
  Description of Artificial Sequence Synthetic oligonucleotide
ctcgagcaatggatctggcgggtctggaggctctggagggtcgaatggctctggag

SEQ ID NO: 14 - DNA, 64 bp, Artificial Sequence
  Description of Artificial Sequence Synthetic oligonucleotide
ggatccgttccctccagacccgccagatcccccagagcctccagagccattcgaccctccagag

SEQ ID NO: 15 - DNA, 46 bp, Artificial Sequence
  Description of Artificial Sequence Synthetic oligonucleotide
cctcgagctctgtgagccagacctccaagctcaccagggccgagac

SEQ ID NO: 16 - DNA, 44 bp, Artificial Sequence
  Description of Artificial Sequence Synthetic oligonucleotide
gggatccgtccacatcagggaagacagtctcggccctggtgagc

SEQ ID NO: 17 - DNA, 33 bp, Artificial Sequence
  Description of Artificial Sequence Synthetic oligonucleotide
ggaaaaaacaaagctcacttctgtgagccagac

SEQ ID NO: 18 - DNA, 33 bp, Artificial Sequence
  Description of Artificial Sequence Synthetic oligonucleotide
gtctggctcacagaagtgagctttgttttttcc

SEQ ID NO: 19 - DNA, 38 bp, Artificial Sequence
  Description of Artificial Sequence Synthetic oligonucleotide
cctcgagcagagcttcaatgacttcacccgggtggtgg

SEQ ID NO: 20 - DNA, 41 bp, Artificial Sequence
  Description of Artificial Sequence Synthetic oligonucleotide
gggatccatcctccccgcccaccacccgggtgaagtcattg

SEQ ID NO: 21 - DNA, 34 bp, Artificial Sequence
  Description of Artificial Sequence Synthetic oligonucleotide
ggaaaaaacaaagctcactcagagcttcaatgac

SEQ ID NO: 22 - DNA, 34 bp, Artificial Sequence
  Description of Artificial Sequence Synthetic oligonucleotide
gtcattgaagctctgagtgagctttgttttttcc

SEQ ID NO: 23 - DNA, 31 bp, Artificial Sequence
  Description of Artificial Sequence Synthetic oligonucleotide
gtgggatccgatgcacacaagagtgaggttg

SEQ ID NO: 24 - DNA, 35 bp, Artificial Sequence
  Description of Artificial Sequence Synthetic oligonucleotide
cacggatccctataagcctaaggcagcttgacttg

SEQ ID NO: 25 - DNA, 18 bp, Artificial Sequence
  Description of Artificial Sequence Synthetic oligonucleotide
caaggagacgggcgctcc

SEQ ID NO: 26 - DNA, 19 bp, Artificial Sequence
  Description of Artificial Sequence Synthetic oligonucleotide
gcccaaggaggggattggc

SEQ ID NO: 27 - DNA, 30 bp, Artificial Sequence
  Description of Artificial Sequence Synthetic oligonucleotide
gtggaattcatggagctgaggccctggttg

SEQ ID NO: 28 - DNA, 38 bp, Artificial Sequence
  Description of Artificial Sequence Synthetic oligonucleotide
cacgcggccgctcactacagccgttgccccgcctccac

SEQ ID NO: 29 - protein, 704 aa, Homo sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFTTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRL

SEQ ID NO: 30 - protein, 25 aa, Artificial Sequence
  Description of Artificial Sequence Synthetic linker peptide
SSGGSGGSGGSGGSGGSGGSGGSGS

SEQ ID NO: 31 - protein, 31 aa, Artificial Sequence
  Description of Artificial Sequence Synthetic linker peptide
SSNGSGGSGGSGGSNGSGGSGGSGGSGGNGS

SEQ ID NO: 32 - protein, 17 aa, Artificial Sequence
  Description of Artificial Sequence Synthetic linker peptide
RAETVFPDVDYVNSTEA

SEQ ID NO: 33 - protein, 14 aa, Artificial Sequence
  Description of Artificial Sequence Synthetic linker peptide
RAETVFPDVDYVNS

SEQ ID NO: 34 - protein, 17 aa, Artificial Sequence
  Description of Artificial Sequence Synthetic linker peptide
RAEAVFPDVDYVNSTEA

SEQ ID NO: 35 - protein, 19 aa, Artificial Sequence
  Description of Artificial Sequence Synthetic linker peptide
RAEAVFPDVDYVNSTEAGS

SEQ ID NO: 36 - protein, 19 aa, Artificial Sequence
  Description of Artificial Sequence Synthetic linker peptide
SVSQTSKLTRAETVFPDVD

SEQ ID NO: 37 - protein, 21 aa, Artificial Sequence
  Description of Artificial Sequence Synthetic linker peptide
SVSQTSKLTRAETVFPDVDGS

SEQ ID NO: 38 - protein, 19 aa, Artificial Sequence
  Description of Artificial Sequence Synthetic linker peptide
SVSQTSKLTRAETVFPDVD

SEQ ID NO: 39 - protein, 21 aa, Artificial Sequence
  Description of Artificial Sequence Synthetic linker peptide
SVSQTSKLTRAETVFPDVDGS

SEQ ID NO: 40 - protein, 22 aa, Artificial Sequence
  Description of Artificial Sequence Synthetic linker peptide
SVSQTSKLTRAETVFPDVDNGS

SEQ ID NO: 41 - protein, 19 aa, Artificial Sequence [sequence omitted]

SEQ ID NO: 42 - protein, 19 aa, Artificial Sequence
  Description of Artificial Sequence Synthetic linker peptide
SVSQTSKLTRTETVFPDVD

SEQ ID NO: 43 - protein, 19 aa, Artificial Sequence
  Description of Artificial Sequence Synthetic linker peptide
SVSQTSKLTRSETVFPDVD

SEQ ID NO: 44 - protein, 19 aa, Artificial Sequence [sequence omitted]

SEQ ID NO: 45 - protein, 19 aa, Artificial Sequence
  Description of Artificial Sequence Synthetic linker peptide
SVSQTSKLTRTEAVFPDVD

SEQ ID NO: 46 - protein, 19 aa, Artificial Sequence
  Description of Artificial Sequence Synthetic linker peptide
SVSQTSKLTRGEAVFPDVD

SEQ ID NO: 47 - protein, 14 aa, Artificial Sequence
  Description of Artificial Sequence Synthetic linker peptide
SKLTRAETVFPDVD

SEQ ID NO: 48 - protein, 14 aa, Artificial Sequence
  Description of Artificial Sequence Synthetic linker peptide
QSFNDFTRVVGGED

SEQ ID NO: 49 - protein, 16 aa, Artificial Sequence
  Description of Artificial Sequence Synthetic linker peptide
QSFNDFTRVVGGEDGS

SEQ ID NO: 50 - protein, 14 aa, Artificial Sequence
  Description of Artificial Sequence Synthetic linker peptide
QSFNDFTRTVGGED

SEQ ID NO: 51 - protein, 14 aa, Artificial Sequence
  Description of Artificial Sequence Synthetic linker peptide
QSFNDFTRLVGGED

SEQ ID NO: 52 - protein, 14 aa, Artificial Sequence
  Description of Artificial Sequence Synthetic linker peptide
QSFNDFTRGVGGED

SEQ ID NO: 53 - protein, 17 aa, Artificial Sequence
  Description of Artificial Sequence Synthetic linker peptide
QSFNDFTRVVGGEDNGS

SEQ ID NO: 54 - protein, 15 aa, Artificial Sequence
  Description of Artificial Sequence Synthetic linker peptide
QSFNDFTRVVGGEDN

SEQ ID NO: 55 - protein, 7 aa, Artificial Sequence
  Description of Artificial Sequence Synthetic linker peptide
RIVGGQE

SEQ ID NO: 56 - protein, 7 aa, Artificial Sequence
  Description of Artificial Sequence Synthetic linker peptide
RLVGGQE

SEQ ID NO: 57 - protein, 7 aa, Artificial Sequence
  Description of Artificial Sequence Synthetic linker peptide
RTVGGQE

SEQ ID NO: 58 - protein, 7 aa, Artificial Sequence
  Description of Artificial Sequence Synthetic linker peptide
RVVGGQE

SEQ ID NO: 59 - protein, 7 aa, Artificial Sequence
  Description of Artificial Sequence Synthetic linker peptide
RAVGGQE

SEQ ID NO: 60 - protein, 7 aa, Artificial Sequence
  Description of Artificial Sequence Synthetic linker peptide
RGVGGQE

SEQ ID NO: 61 - protein, 16 aa, Artificial Sequence
  Description of Artificial Sequence Synthetic linker peptide
PERGDNNLTRIVGGQE

SEQ ID NO: 62 - protein, 19 aa, Artificial Sequence
  Description of Artificial Sequence Synthetic linker peptide
SVSQTSKLTRAETVFPDVD

SEQ ID NO: 63 - protein, 15 aa, Artificial Sequence
  Description of Artificial Sequence Synthetic linker peptide
QSFNDFTRVVGGEDN

SEQ ID NO: 64 - protein, 19 aa, Artificial Sequence
  Description of Artificial Sequence Synthetic linker peptide
SVSQTSKLTRAETVFPDVD

SEQ ID NO: 65 - protein, 16 aa, Artificial Sequence
  Description of Artificial Sequence Synthetic linker peptide
KRNASKPQGRIVGGKV

SEQ ID NO: 66 - protein, 16 aa, Artificial Sequence
  Description of Artificial Sequence Synthetic linker peptide
PEEPQLRMKNNEEAED

SEQ ID NO: 67 - protein, 19 aa, Artificial Sequence
  Description of Artificial Sequence Synthetic linker peptide
DNSPSFIQIRSVAKKHPKT

SEQ ID NO: 68 - protein, 20 aa, Artificial Sequence
  Description of Artificial Sequence Synthetic linker peptide
LSKNNAIEPRSFSQNSRHPS

SEQ ID NO: 69 - protein, 21 aa, Artificial Sequence
  Description of Artificial Sequence Synthetic linker peptide
DEDENQSPRSFQKKTRHYFIA

SEQ ID NO: 70 - protein, 16 aa, Artificial Sequence
  Description of Artificial Sequence Synthetic linker peptide
SPHVLRNRAQSGSVPQ

SEQ ID NO: 71 - protein, 24 aa, Artificial Sequence
  Description of Artificial Sequence Synthetic linker peptide
PEEPQLRMKNNEEAEDYDDDLTDS

SEQ ID NO: 72 - protein, 31 aa, Artificial Sequence
  Description of Artificial Sequence Synthetic linker peptide
DDDNSPSFIQIRSVAKKHPKTWVHYAAEEED

SEQ ID NO: 73 - protein, 28 aa, Artificial Sequence
  Description of Artificial Sequence Synthetic linker peptide
LSKNNAIEPRSFSQNSRHPSTRQKQFNA

SEQ ID NO: 74 - protein, 22 aa, Artificial Sequence
  Description of Artificial Sequence Synthetic linker peptide
DEDENQSPRSFQKKTRHYFIAA

SEQ ID NO: 75 - protein, 32 aa, Artificial Sequence
  Description of Artificial Sequence Synthetic linker peptide
DYGMSSSPHVLRNRAQSGSVPQFKKVVFQEFT

SEQ ID NO: 76 - protein, 24 aa, Artificial Sequence
  Description of Artificial Sequence Synthetic linker peptide
DIYDEDENQSPRSFQKKTRHYFIA

SEQ ID NO: 77 - protein, 17 aa, Artificial Sequence
  Description of Artificial Sequence Synthetic linker peptide
DNSPSFIQIRSVAKKHP

SEQ ID NO: 78 - protein, 20 aa, Artificial Sequence
  Description of Artificial Sequence Synthetic linker peptide
LSKNNAIEPRSFSQNSRHPS

SEQ ID NO: 79 - protein, 36 aa, Artificial Sequence
  Description of Artificial Sequence Synthetic linker peptide
SVSQTSKLTRAETVFPDVTQPERGDNNLTRIVGGQE

SEQ ID NO: 80 - protein, 23 aa, Artificial Sequence
  Description of Artificial Sequence Synthetic linker peptide
SKLTRAETVFPDNNLTRIVGGQE

SEQ ID NO: 81 - protein, 27 aa, Artificial Sequence
  Description of Artificial Sequence Synthetic linker peptide
RAETVFPDVTQPERGDNNLTRIVGGQE

SEQ ID NO: 82 - protein, 22 aa, Artificial Sequence
  Description of Artificial Sequence Synthetic linker peptide
RAETVFPERGDNNLTRIVGGQE

SEQ ID NO: 83 - protein, 32 aa, Artificial Sequence
  Description of Artificial Sequence Synthetic linker peptide
SVSQTSKLTRAETVFPDVDYVNNLTRIVGGQE

SEQ ID NO: 84 - protein, 30 aa, Artificial Sequence
  Description of Artificial Sequence Synthetic linker peptide
SVSQTSKLTRAETVFPDVDNNLTRIVGGQE

SEQ ID NO: 85 - protein, 30 aa, Artificial Sequence; identical to SEQ ID NO: 84
  Description of Artificial Sequence Synthetic linker peptide

SEQ ID NO: 86 - protein, 52 aa, Artificial Sequence
  Description of Artificial Sequence Synthetic linker peptide
SVSQTSKLTRAETVFPDVDYVNSTEAETILDNITQSTQSFNDFTRVVGGEDA

SEQ ID NO: 87 - protein, 32 aa, Artificial Sequence
  Description of Artificial Sequence Synthetic linker peptide
SVSQTSKLTRAETVFPDVQSFNDFTRVVGGED

SEQ ID NO: 88 - protein, 32 aa, Artificial Sequence
  Description of Artificial Sequence Synthetic linker peptide
SVSQTSKLTRAETVFPDVDSFNDFTRVVGGED

SEQ ID NO: 89 - protein, 32 aa, Artificial Sequence
  Description of Artificial Sequence Synthetic linker peptide
SVSQTSKLTRAETVFPDVNASKPQGRIVGGKV

SEQ ID NO: 90 - protein, 32 aa, Artificial Sequence
  Description of Artificial Sequence Synthetic linker peptide
SVSQTSKLTRAETVFPDVNASKPQGRLVGGKV

SEQ ID NO: 91 - protein, 32 aa, Artificial Sequence
  Description of Artificial Sequence Synthetic linker peptide
SVSQTSKLTRAETVFPDVNASKPQGRTVGGKV

SEQ ID NO: 92 - protein, 19 aa, Artificial Sequence
  Description of Artificial Sequence Synthetic linker peptide
SVSQTSKLTRAETVFPDVD

SEQ ID NO: 93 - protein, 666 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTSVSQTSKLTRAETVFPDVDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 7 aa, Artificial Sequence
  Description of Artificial Sequence Synthetic linker peptide
GGGGGGV

SEQ ID NO: 95 - protein, 24 aa, Artificial Sequence
  Description of Artificial Sequence Synthetic linker peptide
SVSQTSKLTRAETVFPDVDGSGGS

SEQ ID NO: 96 - protein, 18 aa, Artificial Sequence
  Description of Artificial Sequence Synthetic linker peptide
SVSQTSKLTRAETVFPDV

SEQ ID NO: 97 - protein, 15 aa, Artificial Sequence
  Description of Artificial Sequence Synthetic linker peptide
QTSKLTRAETVFPDV

SEQ ID NO: 98 - protein, 13 aa, Artificial Sequence
  Description of Artificial Sequence Synthetic linker peptide
SKLTRAETVFPDV

SEQ ID NO: 99 - protein, 16 aa, Artificial Sequence
  Description of Artificial Sequence Synthetic linker peptide
SVSQTSKLTRAETVFP

SEQ ID NO: 100 - protein, 15 aa, Artificial Sequence
  Description of Artificial Sequence Synthetic linker peptide
SVSQTSKLTRAETVF

SEQ ID NO: 101 - protein, 12 aa, Artificial Sequence
  Description of Artificial Sequence Synthetic linker peptide
QTSKLTRAETVF

SEQ ID NO: 102 - protein, 10 aa, Artificial Sequence
  Description of Artificial Sequence Synthetic linker peptide
SKLTRAETVF

SEQ ID NO: 103 - protein, 13 aa, Artificial Sequence
  Description of Artificial Sequence Synthetic linker peptide
SVSQTSKLTRAET

SEQ ID NO: 104 - protein, 10 aa, Artificial Sequence
  Description of Artificial Sequence Synthetic linker peptide
QTSKLTRAET

SEQ ID NO: 105 - protein, 8 aa, Artificial Sequence
  Description of Artificial Sequence Synthetic linker peptide
SKLTRAET

SEQ ID NO: 106 - protein, 18 aa, Artificial Sequence
  Description of Artificial Sequence Synthetic linker peptide
QTSKLTRAETVFPDVDGS

SEQ ID NO: 107 - protein, 16 aa, Artificial Sequence
  Description of Artificial Sequence Synthetic linker peptide
SKLTRAETVFPDVDGS

SEQ ID NO: 108 - protein, 13 aa, Artificial Sequence
  Description of Artificial Sequence Synthetic linker peptide
QSFNDFTRVVGGE

SEQ ID NO: 109 - protein, 18 aa, Artificial Sequence
  Description of Artificial Sequence Synthetic linker peptide
PERGDNNLTRIVGGQEGS

SEQ ID NO: 110 - protein, 15 aa, Artificial Sequence
  Description of Artificial Sequence Synthetic linker peptide
PERGDNNLTRIVGGQ

SEQ ID NO: 111 - protein, 11 aa, Artificial Sequence
  Description of Artificial Sequence Synthetic linker peptide
DNNLTRIVGGQ

SEQ ID NO: 112 - protein, 11 aa, Artificial Sequence
  Description of Artificial Sequence Synthetic linker peptide
ASKPQGRIVGG

SEQ ID NO: 113 - protein, 5 aa, Artificial Sequence Sequence
  Description of Artificial Sequence Synthetic peptide
GGSGS

SEQ ID NO: 114 - protein, 39 aa, Artificial Sequence Sequence
  Description of Artificial Sequence Synthetic peptide
KEKTKLTSSGGSGGSGGSGGSGGSGGSGGSGSDAHKSEV

SEQ ID NO: 115 - protein, 45 aa, Artificial Sequence Sequence
  Description of Artificial Sequence Synthetic peptide
KEKTKLTSSNGSGGSGGSGGSNGSGGSGGSGGSGGNGSDAHKSEV

SEQ ID NO: 116 - protein, 35 aa, Artificial Sequence Sequence
  Description of Artificial Sequence Synthetic peptide
KEKTKLTSVSQTSKLTRAETVFPDVDGSDAHKSEV

SEQ ID NO: 117 - protein, 30 aa, Artificial Sequence Sequence
  Description of Artificial Sequence Synthetic peptide
KEKTKLTQSFNDFTRVVGGEDGSDAHKSEV